(19) United States
(12) Patent Application Publication
(10) Pub. No.: US 2017/0239036 A1
(43) Pub. Date: Aug. 24, 2017

(54) IMPLANTABLE DEVICES COMPRISING GRAFT MEMBRANES

(71) Applicant: AMNIS THERAPEUTICS LTD., Or Akiva (IL)

(72) Inventors: Shahar COHEN, Kiryat Bialik (IL); Noam SHAMAY, Moshav Beit Uziel (IL); Zvi BOMS, Beit Hanania (IL)

(21) Appl. No.: 15/506,864

(22) PCT Filed: Aug. 27, 2015
(86) PCT No.: PCT/IL2015/050865

§ 371 Date: Feb. 27, 2017

Related U.S. Application Data
(60) Provisional application No. 62/188,875, filed on Jul. 6, 2015.
(60) Provisional application No. 62/042,306, filed on Aug. 27, 2014.

(51) Int. Cl. A61F 2/07; A61L 27/36; A61F 2/88

(52) U.S. Cl. CPC A61F 2/07; A61F 2/88; A61L 27/3604; A61L 27/3683; A61F 2210/0014; A61F 2230/0069

(57) ABSTRACT

The invention provides implantable devices and membranes suitable for implantation in a body lumen, as well as methods and processes for their preparation.

### TECHNOLOGICAL FIELD

The invention is in the field of medical devices comprising graft membranes suitable for deployment in a lumen, such as a blood vessel of a patient.

### BACKGROUND ART

References considered to be relevant as background to the presently disclosed subject matter are listed below:
[1] Jiang et al, IEEE/ICME International Conference on Complex Medical Engineering 2007, 1961-1964
[2] WO 2010/101780
[3] WO 2006/073626

Acknowledgement of the above references herein is not to be inferred as meaning that these are in any way relevant to the patentability of the presently disclosed subject matter.

### BACKGROUND

Covered stents are typically tubular expandable devices which are partially, at times fully covered by a single or multiple layers of covering material that are implanted within a patient's vasculature or other body lumen in order to reconstruct blood vessels or body lumens for restoring blood flow or other body fluids therethrough [1].

Such stents are typically structured from a metallic or plastic scaffold, which is expandable or deployable within the lumen after their insertion [2-3]. The stents are covered, or encased, in a synthetic or biological tissue in order to provide a jacket, for enabling restoration of a damaged blood vessel wall (in case of perforation or an aneurysm). Covered stent are often used to treat intravascular perforations or dissections as well as for trapping plaque between the blood vessel wall and the stent coverage hereby preventing it from being released into the blood stream.

In order to enable their deployment in the lumen as well as provide support to the cover tissue, the scaffold is often a complex engineered structure, which is both complicated and expensive to produce. In addition, as the stent is often left within the lumen for prolonged periods of time (sometimes for years), the rich in metal or plastic scaffolds may cause undesired reactions in the patient's tissues surrounding the stent, resulting in rejection of the stent or inflammation in the vicinity of the stent.

### GENERAL DESCRIPTION

The present disclosure provides implantable devices having a minimal and simple frame structure, which provides sufficient mechanical support for graft membranes and the lumen into which the device is implanted, while minimizing the undesired side-effects associated with the robust structure of conventional stents.

The inventors of the present invention have come to the realization that 2-dimensional frames, to which a suitable membrane is attached, can be twisted or deformed in order to obtain a 3-dimensional implantable device suitable for implantation into tubular cavities or lumens. The devices of the present disclosure are thus structured from a frame element supporting a relatively large-surfaced membrane (relative to the frame), that once deformed into its 3-dimensional shape, provides maximal contact between the membrane and the lumen to be treated.

Furthermore, the design of the implantable device permits switching from the 2D to the 3D configuration with substantially no surface tension or trauma caused to the membrane, thereby maintaining the integrity and mechanical properties of the membrane. The 2D configuration of the device allows its ease of assembly or preparation, while its 3D configuration provides for proper deployment of the device in the lumen to be treated and suitable mechanical support therein. In addition, the dimensions of the frame element render the frame with improved flexibility, such that transition between 2D and 3D configurations, as well as deployment in the lumen is easier.

Thus, in one of its aspects, the present disclosure provides an implantable device for positioning a membrane in a body lumen, the device comprising a frame element and a membrane attached to the frame element, the device having a first, 2-dimensional spatial configuration, and a second, 3-dimensional spatial configuration, the device being switchable between the first and second configurations.

The device of this disclosure assumes two configurations. The 2-dimensional (2D) configuration refers to a substantially planar spatial configuration, in which the device has one dimension significantly smaller than its other dimensions. When the device is in its 2D configuration, its planar dimensions (i.e. breadth and width) are often at least 10, 20, 30 or even 50-folds larger than the device's thickness. For deployment, the device is formed into a 3-dimensional configuration, such that the device assumes a voluminous spatial shape that enables the device to provide mechanical support to the lumen section into which the device is inserted or implanted.

In some embodiments, the device may include one or more loops, lassos, or sutures to facilitate positioning or removal of the device during or after implantation.

The frame element is, by some embodiments, a closed-loop wire. Namely, the frame element is made of a wire and defines a substantially closed geometrical shape. In some embodiments, the frame element has a polygonal, oval or round 2-dimentional (2D) shape. The term substantially closed shape means to denote that the wire, when in its 2D configuration, forms a planar loop, which may have a single opening. In other embodiments, the frame element has a completely closed-loop 2D configuration.

The term wire denotes an elongated piece, which has one of its dimensions substantively larger than the other two. The wire may be a single, homogenous metal or polymeric material or composition, or a combination of suitable construction materials. The wire may be constituted by a single filament of material or two or more filaments or strands, being made of the same or different materials, fabricated by twisting, braiding, welding, extruding, or otherwise machining together one or more filaments to form the wire.

The thickness of the device in the 2D configuration is predominantly determined by the thickness (or diameter) of the wire. According to some embodiments, the wire has a diameter of between about 0.001 and 0.018 inch. Alternatively, the wire may have a flat rectangular cross-section, having a thickness of between about 0.001 and 0.018 inch. In some embodiments, the diameter (or thickness) of the wire is between about 0.001 and 0.01 inch.

In other embodiments, the frame element may be formed from sheets or tubular blanks. In such cases, the frame element may be fabricated by laser cutting, chemical etching, high-pressure water etching, mechanical cutting, cold stamping, electro discharge machining, or any suitable fabrication method.

In some embodiments, the device has a substantially tubular 3-dimentional spatial configuration when it assumes its second configuration. The term substantially tubular refers to an elongated, hollow cylinder, having a polygonal, oval or circular cross section (taken perpendicular to the cylinder's longitudinal axis). In some embodiments, the tube's cross-section is a circle. The tube is often uniform in diameter, although its diameter may vary along its longitudinal axis (depending on the local geometry of the lumen to be treated).

The device is designed to be switchable from the first (2D) configuration, typically used for manufacturing and storage, to the second (3D) configuration prior to implantation. The switch (or any lingual variation thereof) is carried out by twisting or deforming the device from the planar configuration to the desired 3D shape, for example, a shape that is suitable for implantation into a lumen or a cavity in a patient's body.

In some embodiments, the device is switchable from said first configuration to said second configuration by folding the device about a longitudinal symmetry axis. In a non-limiting example, the device has a 2D disc configuration, and a tubular 3D configuration obtained by rolling the disc into a tube shape about the diameter of the disc.

In other embodiments, when the device is in its second configuration, the frame is substantially helical. For this purpose, the frame element may have, as mentioned above, an opening that permits it to assume a helical 3D shape once the device is deployed.

Although minimally coming into contact with the tissue of the patient, if at all, the frame element is made of biocompatible materials. In some embodiments, the biocompatible material is selected from a metal or a metal alloy, a polymer or a carbon-based material.

According to some embodiments, the biocompatible material may be a shape memory alloy or a shape-memory polymer. The term shape memory material refers to a material or composition of matter (such as an alloy) which can be deformed to various shapes and can return to assume a permanent pre-deformed shape upon exposure to a certain trigger. Namely, the frame element may have its pre-deformed (permanent) shape of the 3D configuration, and temporarily deformed into its 2D configuration for each of construction. Once heated or mechanically triggered, the shape memory material from which the frame is constructed will switch from the 2D to the 3D configuration due to the shape-memory properties of its constructions material. In some embodiments, the switch between the first configuration and second configuration is induced by heating or by application of force onto the device.

Non-limiting examples of materials from which the frame element may be prepared are stainless steel, nitinol, MP35N, gold, tantalum, platinum or platinum iridium, niobium, tungsten, iconel, ceramic, nickel, titanium, stainless steel/titanium composite, cobalt, chromium, cobalt/chromium alloys, magnesium, aluminum, or other biocompatible metals and or composites or alloys. Examples of other materials that may be used to form the frame element include carbon or carbon fiber, cellulose acetate, cellulose nitrate, silicone, polyethylene terephthalate, polyurethane, polyamide, polyester, polyorthoester, polyanhydride, polyether sulfone, polycarbonate, polypropylene, ultra high molecular weight polyethylene, polytetrafluoroethylene, or another biocompatible polymeric material, or mixtures or copolymers of these; polylactic acid, polyglycolic acid or copolymers thereof, a polyanhydride, polycaprolactone, polyhydroxybutyrate valerate or another biodegradable polymer, or mixtures or copolymers of these, a protein, an extracellular matrix component, collagen, fibrin, or another biologic agent, or any suitable mixture thereof. The term biodegradable is art-recognized, and includes polymers, composites and formulations comprising thereof, including those described herein, that are intended to degrade during in vivo use, such as implantation.

To enable imaging during implantation, as well as tracing of the device post-implantation, the frame element may comprise one or more markers, such as radio-opaque markers suitable for fluoroscopic visualization.

To the frame element, a membrane is attached, thereby forming the device. The membrane is attached to the frame element such that the membrane is stretched over the entire area enclosed by the frame element. Namely, as in its 2D configuration, the frame element forms a planar, substantially closed-loop, such loop encloses a defined planar area. The membrane is attached to the frame element such that the entire area enclosed by the frame element is covered by the membrane. The membrane may by a single layer, i.e. stretching over the area enclosed by the frame element from one side thereof, or a bi-layer—thereby covering the frame element from both its sides and actually encasing the frame element.

In some embodiments, the membrane is made of a biocompatible material, which may be synthetic or of a biological source. Exemplary suitable synthetic biocompatible materials are siliconic polymers, polyurethanes, thermoplastic elastomers, polyolefin elastomers, polyethylene, polytetrafluoroethylene (PTFE), nylon, and copolymers and/or combinations thereof. In some embodiments, the membrane comprises porous fluoropolymer materials, and in particular, expanded polytetrafluoroethylene (ePTFE) materials. Where the device is to be implanted in low pH environments, such as gastric or bail acid environments, the membrane is selected to resist low pH values.

In other embodiments, the membrane is an animal tissue graft membrane, which may be a tissue graft that has been treated as described further below. The tissue graft may be isolated or harvested from various types of organs or tissues, and is not limited to any particular species or type of tissue or organ. According to some embodiments, the animal tissue graft is prepared from prenatal tissue, postnatal tissue, or adult tissue. According to other embodiments, the membrane is prepared from a human tissue graft.

In some embodiments, the tissue graft is prepared from a pericardial tissue, a placental tissue, an amniotic membrane and an umbilical-cord tissue. According to some other embodiments, the membrane is prepared from human placenta tissue. It is noted that the membrane may be autologous, allogenic or xenogenic to the treated patient.

As noted above, the device is designed as to not damage the membrane during transition from 2D to 3D configuration. In order to prevent undesired tearing or uneven spread of tension forces during this transition, in some embodiments the membrane is selected or pre-treated to resemble an elastomer in its mechanical properties. In some embodiments, the membrane is treated to have a secant tensile modulus at 20% elongation (E₂₀, ε=0.2) of at most 25 N/mm². The term secant modulus is often used when referring to elastomers, and means to denote the tensile modulus of the membrane at a given elongation; namely, the secant modulus is the slope of a linear line connecting the point (0,0) and the stress value at a given elongation plotted on the tensile stress-strain curve of the membrane. Meaning, that the secant modulus is calculated from the stress-strain curve obtained by a tensile stress by dividing the measured stress obtained for 20% elongation by 0.2. In some embodiments, the secant modulus at 20% elongation (E₂₀, ε=0.2) ranges between about 0.5 and 25 N/mm². In other embodiments According to other embodiments, the E₂₀ is between about 1 and 25 N/mm², between about 3 and 25 N/mm², between about 5 and 25 N/mm², or even between about 7 and 25 N/mm². Such elastomeric mechanical properties allow the membrane to elastically deform during transition between the 2D and 3D configurations of the device without significant mechanical damage and or exposure to undesired mechanical loads.

Devices of this disclosure are suitable for treating various body lumens and cavities, in which tissue-wall restoration is required, for example airways, blood vessels, urinary tract, gastrointestinal tract and biliary tract. As these lumens vary in size, the device of the present disclosure enables tailoring of the desired dimensions, depending on the dimensions of the lumen to be treated. For example, when intended for implantation in the esophagus, the device may have a 2D disc shape with a diameter of between about 5 and 15 cm (centimeters). Once deformed into the 3D configuration, the resultant tube thus has a length of between about 5 and 15 cm, with a diameter ranging between about 15 and 25 mm (millimeters). In another example, when designed for impanation in the colon, the device may have a length of between about 5 and 15 cm and a diameter ranging between about 20 and 25 mm.

In general, the device may have a length ranging between about 10 mm and 200 mm, and a diameter between about 2 mm and 60 mm.

In another aspect, this disclosure provides a method for obtaining an implantable device as described herein, the method comprising attaching a membrane to a frame element when the device is in its first, 2D configuration, such that the membrane is stretched over the entire area enclosed by the frame.

The term attach (or any lingual variation thereof) means to denote fixing membrane to the frame element in a manner that prevents detachment or peeling of the membrane from the frame element. The method of attachment depends on the type of membrane. In some embodiments, the membrane is made of a non-biological biocompatible material, and attachment of the membrane to the frame element may be carried out by spraying, dipping, painting, brushing, padding, mechanical anchoring, gluing, suturing, or any combination thereof. In other embodiments, wherein the membrane is prepared from an animal tissue graft, the attachment of may be carried out by gluing, suturing, mechanical anchoring, or any combination thereof. In preferred embodiments, the membrane is attached to the frame element by gluing, for example utilizing one or more adhesives, such as medical grade glues (e.g. cyanoacrylate adhesives).

In some other embodiments, the frame element comprises two concentric closed-loop wires, and the membrane is stretched between these two concentric loops (similar to stretching fabric in an embroidery hoop). In such a configuration, the outer wire may comprise tightening means that will allow adjustment of the space formed between the concentric wires as well as the tension applied onto the membrane.

According to some embodiments, the frame element may be pre-treated by polishing, electropolishing, cleaning and/or priming prior to attachment of the membrane.

In further embodiments, the membrane may be pre-treated prior to attachment to the frame element to render it with the desired mechanical properties. In embodiments where the membrane is tissue graft membrane, the membrane may be treated by de-cellularization, thinning (i.e. reducing thickness) and/or a pre-treatment process described herein.

In some embodiments, the animal graft tissue is pre-treated by a de-cellularization process prior to its attachment to the frame element. De-cellularization is a process of removing cells from graft tissue, leaving substantially only the extracellular matrix (ECM) of the graft tissue. Namely, a graft tissue that was de-cellularized will result in a membrane constituted almost entirely by ECM, and being substantially devoid of cellular matter. The term substantially devoid means to denote at most 20% cellular matter, at time at most 10% cellular matter, or even less than 5% cellular matter. An exemplary, non-limiting, de-cellularization process may involve treating the graft tissue with a 0.1% sodium dodecyl sulfate (SDS) solution under shaking conditions in a water-ice bath for 24 hours, followed by washing with distilled water.

In other embodiments, in order to obtain the desired mechanical properties, the graft tissue may be pre-treated by a process comprising immersing the tissue graft in an aqueous solution of a cross-linking agent (such as glutaraldehyde), and washing the tissue graft with a 0.9% saline solution and/or distilled water. In some embodiments, the solution may comprise 0.01-0.4% (v/v) of the cross-linking agent. In other embodiments, the immersion in the glutaraldehyde solution is carried out for between about 30 second and 30 minutes.

Biological tissues require fixation or stripping (e.g. by de-cellularization) of the donor cellular matter prior to their implantation in the host, in order to prevent immune rejection or inflammatory response. Traditional tissue fixating processes, which require a long exposure to fixatives or cross-linkers, often render the graft tissues too stiff to be utilized in devices of the invention. Moreover, traditional fixation methods are typically time consuming, as these involve immersion of at least a few hours (usually at least 24 hours) immersion in fixative solutions. Such typically result in substantial cross-linking or stiffening of the elastin and collagen fibers in the graft tissue, significantly reducing its flexibility and elasticity. The long exposure to fixation agents are often associated with accelerated calcification of the membrane once implanted, poor remodeling that hinders cell attachment and tissue growth, as well as cytotoxicity often preventing tissue healing in the vicinity of the implantable device.

The inventors of the present invention have surprisingly found that short immersion in low-concentration fixative solution is sufficient for obtaining the membrane's desired mechanical properties together with sufficient fixation of the tissue's cellular matter without significantly altering its natural structure and biological properties, while reducing the likelihood or undesired calcification and cytotoxic effects.

Thus, in another of its aspects, the disclosure provides a process of preparing a membrane suitable for implanting into a body lumen, the process comprising:
(a) providing an animal tissue graft;
(b) immersing the tissue graft in an aqueous solution comprising 0.01-0.4% (v/v) at least one cross-linking agent for between about 30 seconds and 30 minutes; and
(c) washing the tissue graft with a physiological solution such as 0.9% saline solution, or with distilled water, thereby obtaining said membrane.

The process of the invention permits gentle treatment of the graft tissue, thereby stabilizing the tissue without overly stiffening it and/or without significantly altering its natural structure and biological properties. The process renders the membrane with superior properties, such as better tensile properties, enhanced biocompatibility, and reduced inflammation, calcification, immune rejection-related responses and better healing responses upon implantation and other toxic effects as compared to synthetic membranes or traditionally treated graft membranes.

According to some embodiments, the at least one cross-linking agent is as least one aldehyde. According to such embodiments, the at least one aldehyde may be selected from glutaraldehyde, formaldehyde, glyceraldehydes, paraformaldehyde, and any combination thereof.

According to other embodiments, the aldehyde is glutaraldehyde.

In some embodiments, the aqueous solution may comprise 0.01, 0.02, 0.03, 0.04, 0.05, 0.06, 0.07, 0.08, 0.09, 0.1, 0.2, 0.25, 0.3, 0.35 or 0.4% (v/v) of said cross-linking agent. In some other embodiments, the solution comprises 0.01-0.3% (v/v) of cross-linking agent (e.g. glutaraldehyde).

The cross-linking agent, by some embodiments, may be a non-aldehyde cross-linking agent.

According to some embodiments, the tissue graft is immersed in the cross-linking agent solution for a period of time between about 30 seconds and 25 minutes, or between about 30 seconds and 20 minutes, or between about 30 seconds and 15 minutes, or between about 30 seconds and 10 minutes, or between about 30 seconds and 5 minutes, or between about 30 seconds and 2.5 minutes, or even between about 30 seconds and 1.5 minutes.

According to other embodiments, the tissue graft is immersed in the cross-linking agent solution for a period of time of 30, 35, 40, 45, 50, 55, 60, 65, 70, 75, 80, 85 or 90 seconds. According to some other embodiments, the tissue graft is immersed in the cross-linking agent solution for about 30-90 seconds, about 30-80 seconds, 30-70 seconds or even 30-60 seconds. According to further other embodiments, the tissue graft is immersed in the cross-linking agent solution for about 35-90 seconds, about 40-90 seconds, or even 45-90 seconds. According to further embodiments, the tissue graft is immersed in the cross-linking agent (e.g. glutaraldehyde) solution for about 40-60 seconds.

In some embodiments, the animal tissue graft to be treated by the process is harvested from prenatal tissue, postnatal tissue, or adult tissue of an animal or a human. In other embodiments, the tissue graft is harvested from a pericardial tissue, a placental tissue, an amniotic membrane and an umbilical-cord tissue. According to some embodiments, the graft tissue is human placenta tissue.

After the graft tissue has been processed by a process of the invention, the resulting membrane, in some embodiments, has a secant tensile modulus at 20% elongation (E₂₀, ε=0.2) of at most 25 N/mm².

Thus, in another aspect of this disclosure, there is provided an animal tissue graft membrane having a secant tensile modulus at 20% elongation (E₂₀, ε=0.2) of at most 25 N/mm².

In some embodiments, the membrane is obtained by de-cellularizing the tissue graft. In other embodiments, the membrane is obtained by the process described herein.

According to some embodiments, the animal tissue graft is harvested from a mammal, which may be human or non-human, and may be harvested from a pericardial tissue, a placental tissue, an amniotic membrane and an umbilical-cord tissue of the animal.

As a person of the art would appreciate, the membranes obtained by a process of this disclosure may be utilized in a variety of implantable devices, as well as used on their own as implantable membranes.

Thus, the present disclosure further provides, in one of its aspects, a stent cover comprising the animal tissue graft membrane as described herein. The term stent cover refers to an outer casing of a stent, the stent being of any shape and form, such that its scaffold structure is substantially enveloped by a cover layer comprising a membrane of the invention.

By another aspect, this disclosure provides a stent comprising a support scaffold and a membrane or a stent cover as described herein attached thereto, the membrane or stent cover constituting a tissue-contacting external surface of the stent.

As already noted above, the stent may have any desired geometry. In some embodiments, the support scaffold is generally tubular. According to such embodiments, the membrane or stent cover may be attached to the support scaffold by a seem-line extending generally parallel to the longitudinal axis of the tubular support scaffold. The seem-line may be bringing together two opposite end portions of the membrane or stent cover and attaching these one to the other by any suitable means known in the art, such as suturing, stapling, gluing, etc.

In another aspect, there is provided a kit, comprising at least one frame element or at least one scaffold structure, at least one membrane as disclosed herein, and means for attaching the membrane to the frame element or the scaffold structure.

In some embodiments, the at least one frame element has a first, 2-dimensional spatial configuration, and a second, 3-dimensional spatial configuration, the frame element being switchable between the first and second configurations. In other embodiments, the scaffold structure is a stent.

The kit may comprise a plurality of frame elements or scaffold structures, having the same or different geometries and dimensions, providing the user to tailor the geometry and dimensions of the implantable device according to its intended use.

Similarly, the kit may comprise a plurality of membranes, the same or different in thickness, dimensions or material. The membranes may be pre-cut to the dimensions suitable for the frames or scaffolds, or provided as sheets for cutting to the desired size and shape.

According to some embodiments, the membrane having a secant tensile modulus at 20% elongation (E₂₀, ε=0.2) of at most 25 N/mm². According to such embodiments, the membrane may be synthetic or prepared from an animal tissue graft.

In embodiments where the membrane is prepared from an animal tissue graft, the tissue graft may be de-cellularized or pre-treated by the process as described herein. In other embodiments, the tissue graft is pre-treated by the process as described herein. The membrane may be provided in dehydrated (i.e. dry) form or in a preservative solution. When in dehydrated form, the kit may further comprise re-hydration solution, such as saline, for rehydrating the dry membrane prior to attachment to the frame or scaffold or prior to implantation.

The attachment means may be any suitable means mentioned hereinabove.

The kit may further comprise instructions for use, such that the implantable device may be prepared by the end user (the medical practitioner) according to the instructions provided or the experience and/or training of the end-user.

The implantable devices, membranes and covered stents of the present disclosure may be inserted or implanted into the patient's lumen to be treated by any suitable means know in the art, such as various endoscopic methods. For example, under the aid of endoscopic and/or fluoroscopic visualization a delivery system containing the implantable device is advanced into the vicinity of the target anatomy. The targeted lumen may be pre-dilated with a balloon catheter or other dilation device, if necessary. In some procedures, the implantable device may be delivered in a compressed state in a low profile delivery system. Once the delivery system is in place, the implantable device may be released from a retaining sheath or the like and positioned in the desired position within the lumen. After implantation, the delivery system may be removed.

As used herein, the term “about” is meant to encompass deviation of ±10% from the specifically mentioned value of a parameter, such as temperature, size, concentration, etc.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1C show various exemplary 3D configurations according to an embodiment of this disclosure, starting from a 2D circular frame.

FIG. 2 shows an exemplary 3D configuration according to an embodiment of this disclosure, starting from a 2D rectangular frame.

FIGS. 3A-3D show process steps in the preparation of an implantable device according to an embodiment of this disclosure.

FIG. 4 shows the implantable device of FIG. 1D, in its deployed position within a simulatory lumen.

FIGS. 5A-5B show histological sections of H&E-stained porcine pericardial tissue treated by glutaraldehyde full fixation (FIG. 5A) and a process of the invention (FIG. 5B).

FIG. 6 presents stress-strain tensile test curves for glutaraldehyde fully fixated membrane (gray) and a membrane treated in a process of the invention (black).

FIG. 7 shows a typical metallic stent covered by a membrane of the invention.

FIGS. 8A-8C present 3-point bending test results for the stent of FIG. 7, covered by a glutaraldehyde fully fixated membrane (gray, FFG) and a membrane treated in a process of the invention (black, pGlut)—% relative applied force (FIG. 8A), % relative curvature upon application of 1N force (FIG. 8B), and % relative curvature upon application of 1.5 N force (FIG. 8C).

FIGS. 9A-9D show host-response to implanted membranes for glutaraldehyde fully fixated membrane (FIGS. 9A-9B) and membranes produced according to a process of the invention (FIGS. 9C-9D).

### DETAILED DESCRIPTION OF EMBODIMENTS

Exemplary Devices

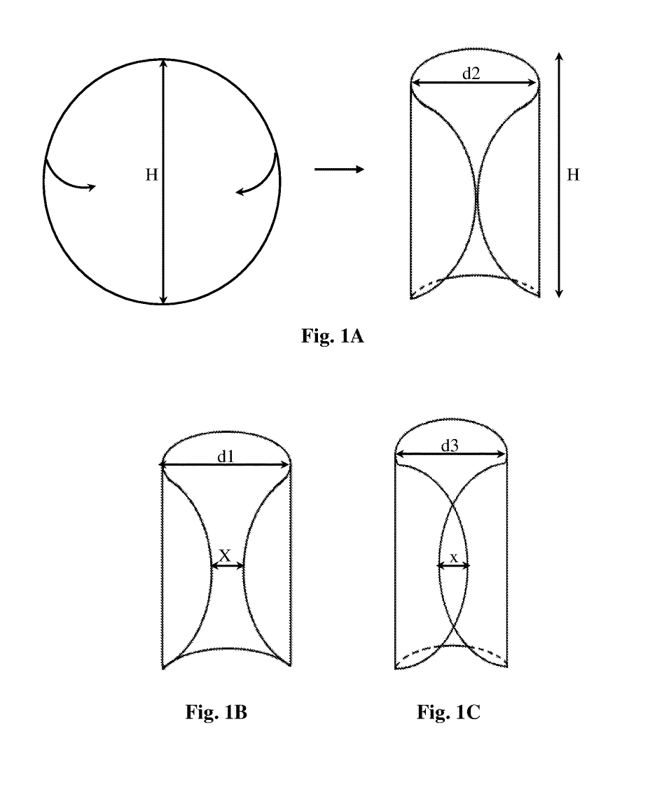

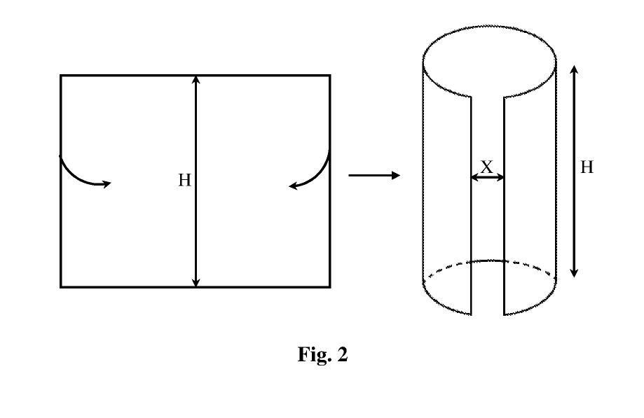

The implantable devices of this disclosure assume at least 2 spatial configurations: a flat, planar 2D configuration and a deployable 3D, voluminous configuration. Examples of such configurations are provided in FIGS. 1A-2.

FIGS. 1A-1C demonstrate various 3D configurations of the implantable device, all starting from a 2D circular frame. These 3D configurations vary in their degree of overlap between sections of the frame, thereby enabling to control the diameter of the tubular 3D configuration. For example, the same 2D disc configuration may be folded to various degrees of overlap of the membrane and frame, thereby resulting in implantable device of the approximately the same length (H), determined by the diameter of the 2D disc, but having different diameters (d1>d2>d3). Such variability enables to insert the device into the lumen when the device has a significantly smaller diameter than the diameter of the lumen (for example in the configuration presented in FIG. 1C), and once positioned in the desired location, the device is allowed to assume a larger diameter (such as that shown in FIG. 1A or 1B).

In addition, the flexibility of the frame allows for variability of the diameter along the tubular device, such that differences in diameters may be obtained along the longitudinal axis of the 3D tubular device. This permits adjustment of the device dimensions to the dimensions of the treated lumen.

FIG. 2 presents a tubular 3D configuration of the device starting from a rectangular 2D planar configuration, thus resulting in a tubular device having a height (H) identical to that of the corresponding 2D planar configuration.

It is of note that the extent of overlap between portions of the device (x) and the distance between the frame edges (X) when the device's edges are not in contact with one another, may be calculated according to the following formula (w is the thickness or diameter of the wire constituting the frame element):

$$\frac{H - x}{π} = {d}_{i} - 2 w$$

$$\frac{H + X}{π} = {d}_{i} - 2 w$$

The extent of overlap x and/or the distance X has an impact on the radial force exerted by the device onto the surrounding lumen, once implanted. Namely, the greater the value of x (and smaller value of X, similarly), the greater the radial force applied by the device onto the lumen. Such variation allows the design and control of the force maintaining the device in position, once implanted, as well as controlling the force applied onto the lumen's tissue that comes into contact with the device.

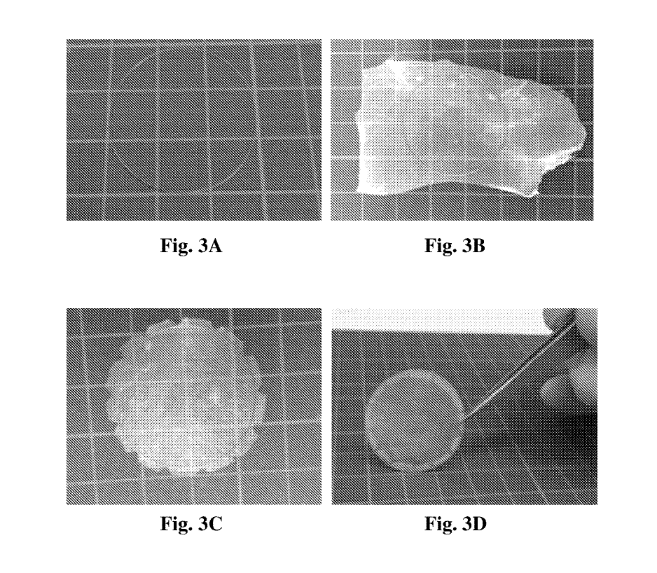

Preparation of an exemplary implantable device according to a method of the present disclosure is demonstrated in FIGS. 3A-3D. A membrane, which may be synthetic or of a biological source (treated or untreated by a process of this disclosure) is placed beneath a circular frame element (as shown in FIGS. 3A-3B). In the exemplary method, the membrane is a human amniotic tissue graft, treated by a process of this disclosure, placed over a stainless steel wire circular frame.

The membrane is then trimmed to the desired dimension and attached to the frame. In this non-limiting example, the membrane is trimmed to form several circumferential flaps (FIG. 3C), which are then folded about the frame element and glued in position by a surgical adhesive (FIG. 3D). Such trimming enables full coverage of the frame element by the membrane without any overlapping or stacking of membrane, as well as application of adhesive only onto the flaps, thereby bonding the membrane flaps to the membrane surface about the frame element to increase bonding strength.

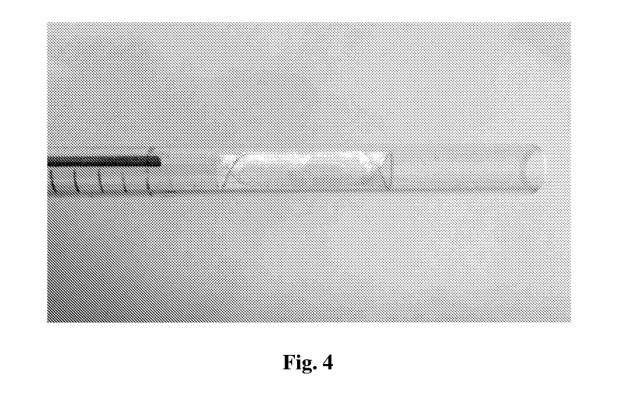

For implantation, the 2D configuration is switched to the 3D configuration by application of mechanical load or heat, whereby the device assumes its permanent 3D configuration, as seen in FIG. 4.

Membrane Preparation

As noted above, it is desired that the membrane used in implantable devices of this disclosure have certain mechanical properties, and preferably have an elastomeric behavior. Due to the inherent non-homogeneity of biological tissues, the inventors have developed a unique pre-treatment process, involving short immersion in low concentration fixative solution, which enables tailoring of the mechanical properties of the graft tissue from which the membrane is prepared, while substantially maintaining its natural structure.

In an exemplary process, porcine pericardial tissue was harvested and thinned to a thickness of 60 μm. The tissue was then treated by immersing the tissue graft in a 0.25% (v/v) aqueous solution of glutaraldehyde for 60 seconds. The tissue was then immediately washed with 0.9% saline (physiological water) and distilled water.

In comparison, a porcine pericardial tissue was treated by a traditional fixation process involving immersing the tissue in a 0.5% (v/v) aqueous solution of glutaraldehyde for 24 hours, and then rinsed with distilled water.

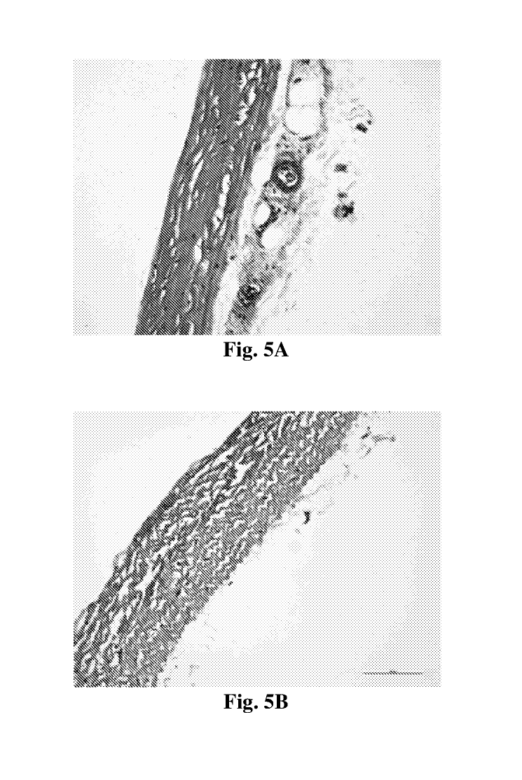

Histological sections were cropped from both samples, stained by hematoxylin and eosin (H&E) stains and visualized. As can be clearly seen from FIGS. 5A-5B, deterioration and cross-linking of the extracellular matrix (mainly collagen and elastin fibers) result in bulky clusters in the traditionally treated tissue (FIG. 5A), while the structure and integrity of the elastin fibers is clearly maintained in the tissue treated by the gentle fixation process of the invention (FIG. 5B). As evident by the results, the treatment process of the invention has no significant effect on the structure of the tissue, however provides sufficient fixation to prevent the undesired side effects associated with the traditional fixation process, as will also be shown below.

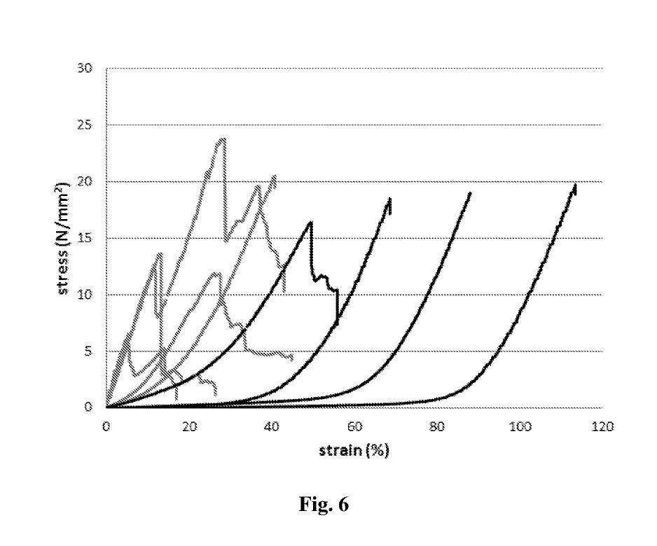

The effect of the fixation process on the mechanical properties of the membrane was assessed by a uni-axial tensile test, in which 15×20 mm samples of the membranes were clamped by suitable metal clamps and subjected to tensile force at a strain rate of 0.5-1 mm/sec. The secant modulus at 20% elongation (E₂₀) was calculated from the stress-strain curves shown in FIG. 6.

As clearly shown in FIG. 6, tissue samples treated by the gentle fixation process showed elastomeric-like mechanical behavior and relatively high elongation to break (at least 50% elongation), while tissues treated by the traditional fixation process failed at significantly lower elongations and demonstrated a stiffer behavior (higher modulus). Some traditionally treated tissues failed well before reaching 20% elongation, and for such, E₂₀ values could not be calculated.

Effect of Membrane Treatment Process on Stent Properties

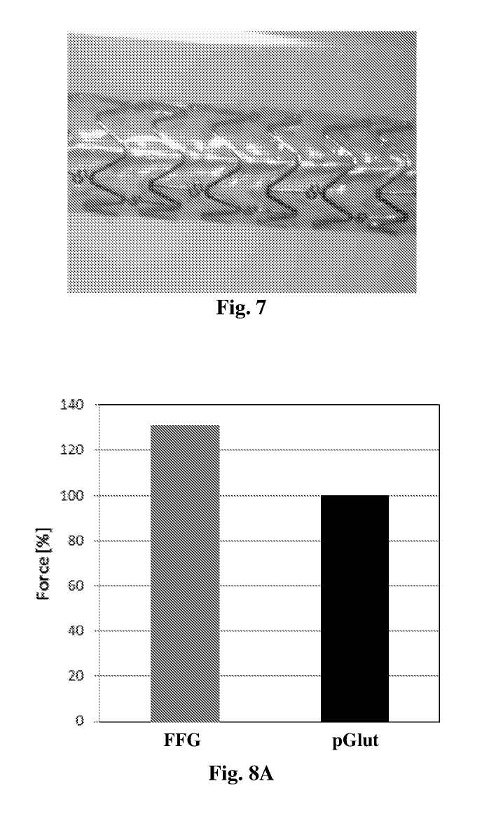

In order to estimate the effect of the membrane on the flexibility of a covered stent, a traditionally-treated membrane (marked “FFG”) and a membrane obtained by a process of the invention (marked “pGlut”), where used to fully cover a metallic stent, as shown in FIG. 7, such that the membrane formed an outer tubular casing of the metallic stent scaffold. The dimensions of the stents were 3×27 mm The thickness of the membranes was 50-60 μm.

The covered stents were subjected to 3-point bending test, in which the stents were positioned horizontally on support legs (distance between the support legs: 11 mm). A force, normal to the stent, is then applied at the midpoint between the supporting legs, thereby bending the sample.

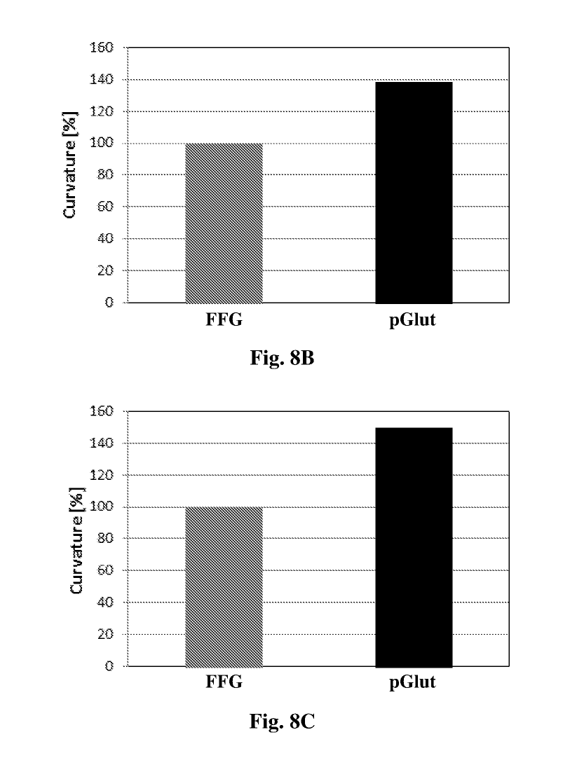

FIG. 8A shows the force required to obtain a 2 mm vertical displacement of the stent at the force-application point. The results are shown relative to the stent covered by the membrane of the invention (i.e. pGlut=100%). As is evident from the results, the force required to obtain a 2 mm displacement for the stent covered by the traditionally-treated membrane is 38% larger than the force required to obtain the same displacement in a stent covered by a membrane of the gentle-fixation process.

Further, the bending radius (curvature) of the covered stents was measured upon application of a constant force. The results shown in FIGS. 8B and 8C show % relative curvature obtained for applied constant force of 1N and 1.5N, respectively, normalized to the curvature of the stent covered with traditionally-treated membrane (i.e. FGG=100%). In both cases, the stents covered with the gentle-fixated membrane enabled obtaining higher curvature (38% and 50% respectively).

The 3-point bending test results clearly indicate that stents covered by membranes of the gentle-fixation process show significantly improved flexibility as compared to the stents covered by traditionally-fixated membranes. This may enable easier implantation and higher structural adaptability to the structure of the lumen in which the stents are to be implanted.

In Vivo Tissue Healing Effect

Assessment of the effect the membrane treatment process is expected to have in vivo, porcine pericardial tissue graft membranes, treated by either the traditional process or the gentle-fixation process, were implanted in healthy mice.

8×8 mm membrane samples prepared in both preparation methods were washed 3 times with saline, and preserved in 0.9% saline and penicillin/streptomycin solution until implantation.

ICR mice were anesthetized and a membrane sample was subcutaneously ectopically implanted into a pocket artificially formed in the dorsal area of the mouse. Each mouse was implanted with both types of membrane samples. After 4 weeks, the mice were humanely euthanized and the tissue-response to the implanted membrane samples was assessed.

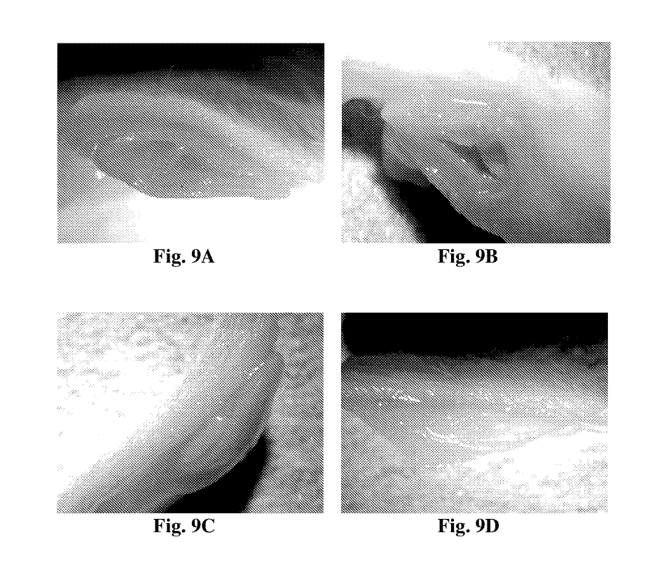

In all mice, a clear and robust inflammatory reaction was observed at and in the vicinity of the traditionally-treated membrane sample, while minimal inflammation was observed at and in the vicinity of the gentle-fixated membrane samples. As is shown is FIGS. 9A-9D, which are cross-sections of the implantation areas, no healing of the pocket tissue was observed in the area in which the traditionally-treated membrane sample was implanted (FIGS. 9A-9B). In comparison, closure of the pocket and tissue healing response was clearly observed in the area into which the gentle-fixated membrane was implanted (FIGS. 9C-9D). Thus, in addition to prevention of host-vs.-graft symptoms, membranes treated in the gentle-fixation of the invention showed the potential of promoting tissue healing in the implantation area. Without wishing to be bound by theory, this may result from the structure of the treated membrane, which remains substantially in its natural (i.e. pre-processed) structure, enabling improved cell adhesion and growth once implanted.

1-55. (canceled)

56. An implantable device for positioning a membrane in a body lumen, the device comprising a frame element and a membrane attached to the frame element, the device having a first, 2-dimensional spatial configuration, and a second, 3-dimensional spatial configuration, the frame element being switchable between the first and second configurations.

57. The device of claim 56, wherein the frame element is a closed-loop wire.

58. The device of claim 56, wherein in the device's first configuration, the frame element has a polygonal, oval or round 2-dimentional shape.

59. The device of claim 56, wherein in the second configuration, the device has a substantially tubular 3-dimentional spatial configuration.

60. The device of claim 56, wherein the device is switchable from said first configuration to said second configuration by folding about a longitudinal symmetry axis.

61. The device of claim 56, wherein in the device's second configuration, the frame is substantially helical.

62. The device of claim 56, wherein the membrane is (i) stretched over at least a portion of the area enclosed by the frame element, or (ii) stretched over the entire area enclosed by the frame element.

63. The device of claim 56, wherein the membrane is an animal or a human tissue graft membrane.

64. The device of claim 63, wherein said animal tissue graft is prepared from prenatal tissue, postnatal tissue, or adult tissue.

65. The device of claim 63, wherein said tissue graft is prepared from a pericardial tissue, a placental tissue, an amniotic membrane and an umbilical-cord tissue.

66. The device of claim 56, wherein said membrane having a secant tensile modulus at 20% elongation (E20) of at most 25 N/mm2, optionally wherein the secant tensile modulus at 20% elongation (E20) is between about 0.5 and 25 N/mm2.

67. A method for obtaining an implantable device of claim 56, comprising attaching a membrane to a frame element when the device is in its first, 2D configuration, such that the membrane is stretched over the entire area enclosed by the frame.

68. The method of claim 67, wherein the membrane is made of an animal tissue graft membrane.

69. The method of claim 68, wherein the attachment of the membrane to the frame element is carried out by gluing, suturing, or mechanical anchoring.

70. The method of claim 68, wherein the membrane is pre-treated by a de-cellularization process.

71. The method of claim 68, wherein the membrane is pre-treated by a process comprising:
(a) immersing the tissue graft in an aqueous solution of at least one cross-linking agent; and
(b) washing the tissue graft with a 0.9% saline solution and/or distilled water.

72. The method of claim 71, wherein said at least one cross-linking agent is at least one aldehyde.

73. The method of claim 71, wherein (i) said solution of step (a) comprises 0.01-0.4% (v/v) of said cross-linking agent; (ii) the immersion in the cross-linking agent solution is carried out for between about 30 seconds and 30 minutes; and/or (iii) the frame element is pre-treated by polishing, electropolishing, cleaning and/or priming prior to attachment of the membrane.

74. A process of preparing a membrane suitable for implanting into a body lumen, the process comprising:
(a) providing an animal or human tissue graft;
(b) immersing the tissue graft in an aqueous solution comprising 0.01-0.4% (v/v) of at least one cross-linking agent for between about 30 seconds and 30 minutes, optionally wherein said at least one cross-linking agent is at least one aldehyde optionally selected from glutaraldehyde, formaldehyde, glyceraldehydes, paraformaldehyde, and any combination thereof; and
(c) washing the tissue graft with a 0.9% saline solution, thereby obtaining said membrane.

75. The process of claim 74, wherein said animal or human tissue graft is harvested from prenatal tissue, postnatal tissue, or adult tissue, optionally wherein said tissue graft is harvested from a pericardial tissue, a placental tissue, an amniotic membrane and an umbilical-cord tissue.
